(19) United States
(12) Patent Application Publication
(10) Pub. No.: US 2026/0253210 A1
(43) Pub. Date: Aug. 27, 2026

(54) SYSTEMS AND METHODS FOR A NOVEL IMAGE-BASED MULTI-OMICS AGING CLOCK FOR THE PREDICTION OF REMAINING LIFESPAN

(71) Applicant: ARIZONA BOARD OF REGENTS ON BEHALF OF THE UNIVERSITY OF ARIZONA, Tuscon, AZ (US)

(72) Inventors: George SUTPHIN, Tucson, AZ (US); Samuel FREITAS, Tucson, AZ (US); Megha PADI, Tucson, AZ (US); Chen CHEN, Tucson, AZ (US)

(21) Appl. No.: 18/879,393

(22) PCT Filed: Jun. 29, 2023
(86) PCT No.: PCT/US2023/069388

§ 371 Date: Dec. 27, 2024

Related U.S. Application Data
(60) Provisional application No. 63/357,425, filed on Jun. 30, 2022.

(51) Int. Cl. G06T 7/00; G16B 40/00; G16H 50/20

(52) U.S. Cl. CPC G06T 7/0012; G16B 40/00; G16H 50/20; G06T 2207/20084; G06T 2207/30101

(57) ABSTRACT

Systems and methods are disclosed for a multi-omics clock designed to predict remaining lifespan in mammals for the purpose of preclinical drug prioritization.

### CROSS-REFERENCE TO RELATED APPLICATIONS

This application claims the benefit of U.S. Provisional App. No. 63/357,425, filed Jun. 30, 2022, the entire contents of which are incorporated herein by reference.

### FIELD OF THE INVENTION

The present invention relates to novel techniques for predicting mammalian lifespans, preferably through the use of a multi-omics clock.

### BACKGROUND OF THE INVENTION

Over the past four decades hundreds of interventions have been identified that extend lifespan in invertebrate model systems of aging. One major bottleneck in translating these interventions into clinical therapies to increase healthy longevity and treat age-associated disease in humans is the high cost in both time and resources associated with conducting longevity studies in preclinical mammalian models. In principle, one solution to this problem is to identify early- to mid-life biomarkers that can predict lifespan and other late-life markers of healthy aging. However, aging is driven by a complex interplay between many molecular and cellular processes, and biomarkers that provide consistent predictive efficacy across interventions, tissues, and disease types have been historically elusive. Recent advances use omics data to develop biomarker panels—tens to thousands of individual molecular measurements (e.g., epigenetic markers, single-gene expression levels, metabolite levels)—to develop predictive “clocks” that can, to some degree, accurately predict biological age or related age-associated metrics. Among many potential uses for these clocks, one application with wide-ranging immediate benefits is in drug discovery and repositioning.

To address the preclinical bottleneck, an ideal clock would be able to accurately predict remaining lifespan in mammals, and robustly capture changes in this metric following short-term treatment with an anti-aging intervention. This timing reflects the likely point in life where a human might expect to receive such an intervention in the clinic. These drugs can then be prioritized for detailed examination in more costly preclinical longevity studies.

### SUMMARY OF THE INVENTION

In certain embodiments, the present invention comprises a server that measures concentrations of one or more biomarkers in a serum sample and receives an image comprising genetic data. Each pixel of the image represents a unique gene. The server then applies a convolutional neural network to the image and the biomarker concentrations to analyze the interactions between the one or more biomarkers and determines a biological status of a mammal from the analysis of the convolutional neural network. The server then outputs one or more treatments for the mammal for the determined biological status.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is an exemplary embodiment of the hardware of the system, which is capable of showing prediction of age difference between paired human peripheral blood mononuclear cell (PBMC) samples by a convolutional neural network (CNN) model trained using RNAseq data;

FIG. 2A is an image of the multilayer perception (MLP) dense network trained on data in a structured format, which outperforms the unstructured format. Red points and blue line indicate performance on 10-fold striated training data. Blue points and purple line indicate performance on blinded validation data held out from the same data set prior to training the models; and

FIG. 2B is an image showing that the present technology outperforms a multilayer perception (MLP) dense network trained on data in an unstructured format (B). Red points and blue line indicate performance on 10-fold striated training data. Blue points and purple line indicate performance on blinded validation data held out from the same data set prior to training the models.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

In certain embodiments, the present invention comprises a novel multi-omics clock designed to predict remaining lifespan in mice for the purpose of preclinical drug prioritization.

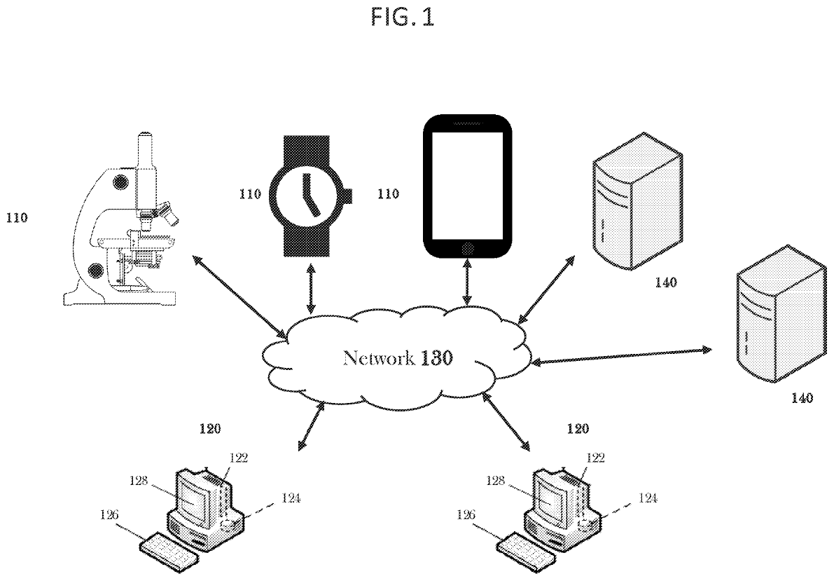

FIG. 1 is an exemplary embodiment of the system of the present invention. In the exemplary system 100, one or more peripheral devices 110 are connected to one or more computers 120 through a network 130. Examples of peripheral devices/locations 110 include smartphones, tablets, wearables devices, medical testing devices, and any other electronic devices that are known in the art that collect and transmit data over a network. The network 130 may be a wide-area network, like the Internet, or a local area network, like an intranet. Because of the network 130, the physical location of the peripheral devices 110 and the computers 120 has no effect on the functionality of the hardware and software of the invention. Both implementations are described herein, and unless specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices/locations 110 and the computers 120 will both include or be attached to communication equipment. Communications are contemplated as occurring through industry-standard protocols such as HTTP or HTTPS.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable devices (e.g. smartphones, smartwatches, tablets). In one embodiment, each of the peripheral devices 110 and each of the computers 120 of the system may have software related to the system installed on it. In such an embodiment, system data may be stored locally on the networked computers 120 or alternately, on one or more remote servers 140 that are accessible to any of the peripheral devices 110 or the networked computers 120 through a network 130. In alternate embodiments, the software runs as an application on the peripheral devices 110, and includes web-based software and iOS-based and Android-based mobile applications.

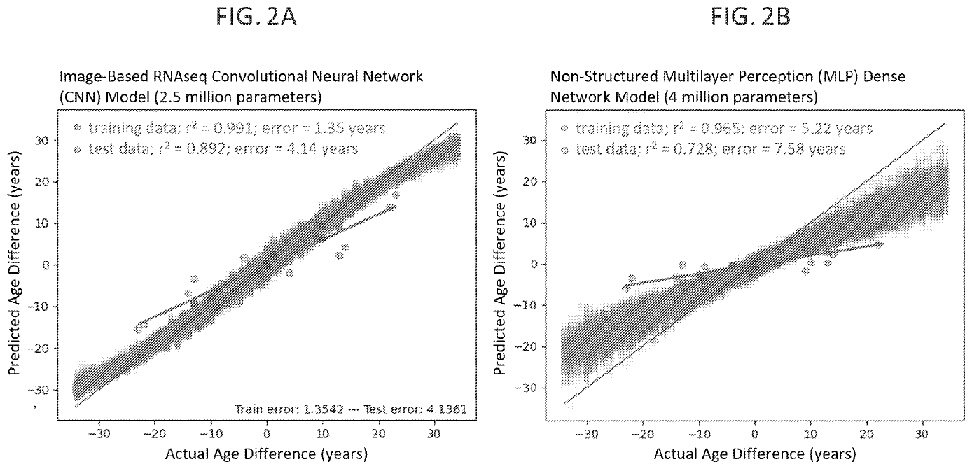

Over the past half decade, artificial intelligence has been employed to improve accuracy of the most common types of clocks, which typically use dense regression models to predict age based on the selected panel of biomarkers. These dense regression-based clocks can predict complex phenotypes like biological age because they consider molecular signatures across a range of processes. However, they can potentially miss interactions between biomarkers that reflect complex interconnectedness of the processes that drive aging. Deep learning methods are capable of considering these complex interactions. At present, the most sophisticated and widely used deep learning methods are in the application of convolutional neural networks (CNNs) to recognize patterns and identify objects in images. While CNNs are not designed for the data structures typically produced by omics technology, recent methods can meaningfully restructure omics data (e.g., transcriptome data) into two-dimensional image-like structures that are compatible with modern CNNs. These tools are used to develop an image-based transcriptomic aging clock using publicly available human gene expression data. Even early, unoptimized versions of this clock are capable of predicting biological age with accuracy on par with current-generation aging clocks (FIG. 2A) and outperforms a parallel clock build using dense networks to predict age based on the same transcriptomic data, but without the novel image-building component (FIG. 2B). We have worked to optimize our methodology for building image-based aging clocks and expand the scope to include multi-omics data.

Beyond the algorithmic details of how the clocks are constructed, there are limitations to the study design used in create current-generation aging clocks. First, while most clocks predict biological age, in many applications the critical phenotype of interest is remaining lifespan. The degree to which the predicted biological age can be extrapolated to remaining lifespan is unclear. Most studies used to build aging clocks use either human data, in which case remaining life expectancy can be measured, but on a timescale that is impractical, or tissue from mice that are sacrificed as part of the sample collection procedure, precluding measurement of remaining lifespan. We are aware of only a single study that predicts both biological age and remaining lifespan in mice using an aging clock based on frailty index (FI); and cumulative health score based on assessment of 29 physiological parameters. The difference between chronological and biological age was not predictive of remaining lifespan. Second, most aging clocks focus on a single “layer” of measurement—DNA methylation, transcriptomics, proteomics, metabolomics, or physiology. Aging results in changes at each of these layers of regulation, and whether any single layer can sufficiently capture age-associated changes in biology to robustly predict either biological age or remaining lifespan in a context-independent fashion, or capture relevant response to intervention is currently unknown. Our second objective was to conduct a mouse aging study to designed for aging clock development by collecting robust multi-omics data in using sample collection methods that are minimally invasive, allowing measurement of longevity and health metrics.

Specific aims and study design. This project accomplishes two primary goals. First, our early pilot testing provides proof-of-principle that organizing omics data into an image can improve accuracy for biological age prediction for machine learning based clocks (FIGS. 2A-2B). This approach is new and utilizes several steps in the clock construction that improve with optimization, which will be the focus of Aim 1 below. Second, data sets that capture more than one omics layer in the same tissue from the same individual animal are rare and none, to our knowledge, capture remaining lifespan. We conduct an aging study in mice that will capture both elements in order to train a multi-omics version of our image-based aging clock that can predict remaining lifespan (Aim 2) and validate the capability of this clock to capture lifespan extension for mice subjected to several aging interventions (Aim 3).

Aim 1. Optimize clock construction using available data. Pilot testing of our image-based aging clock using publicly available RNAseq data from a compiled meta-analyzed dataset including 3,060 samples representing more than 10 distinct human tissues from across the age spectrum shows improved accuracy over a non-image-based approach even without optimization (FIGS. 2A-2B). Here we optimize two steps in the image-based clock construction process: building the imaging from omics data (Aim 1.1) and designing the structure of the machine learning algorithm used to make predictions from the constructed image (Aim 1.2). We use three available data sets to optimize clocks for prediction of biological age. The Human Genotype-Tissue Expression (GTEx) Project has RNAseq data from 980 samples each representing up to 21 major human tissues, including 670 from whole blood. We use this data as our primary tool to build an optimized image-based transcriptomic clock. We validate our optimized clock on a second publicly available data set with 3,060 samples from more than 10 human tissues, including 273 from blood. We use available datasets 19-21 containing RNAseq, shotgun proteomics, and DNA methylation in kidney and liver from a set of 188 diversity outbred (DO) mice at 6, 12, and 18 months of age to optimize the image building step in the context of multi-omics data (Aim 1.2).

Aim 1.1. Optimize image-building algorithm for single-layer and multi-layer omics data. The basic concept in constructing an image from omics data can be illustrated using RNAseq as an example. An image is constructed for each sample, with each gene represented by a pixel. The pixel intensity reflects the gene's expression level in that sample, and the pixel position assigned to each gene is algorithmically determined based on the correlation in expression between genes across samples. To prepare data for image construction, raw RNAseq counts will be normalized across samples using TMM and voom in the R package edgeR. We then combine the training (GTEx) and test datasets and adjust for batch effects using ComBat. That step allows the CNN model to be transferrable between transcriptomic datasets that were generated under different conditions. Dimensionality reduction (PCA and UMAP) and hierarchical clustering will be used to evaluate the success of batch adjustment. Finally, the vector of normalized, log-transformed gene expression values for each sample will be scaled to the range 0-1 before pixel intensities and positions are computed. The high-level process for constructing our current image-based aging clock comprises the following steps:
1. Select features. Our first-order selection is for features that correlate with remaining lifespan using the Spearman correlation and estimated maximal information coefficient (MICe), which captures both linear and more complex (e.g., U-shaped) correlations. The number of features that we select is determined by optimized image size, which in turn is determined by empirical testing.
2. Construct images. Using the Image Generation for Tabular Data (IGTD) algorithm, we organize tabular transcriptomics data into a 2-dimentional image based on Euclidian distance clustering of each feature into a singular point using correlation between gene expression values across samples.
3. Train CNN. The base network is a multi-layer CNN head with a dense body attached to a regression layer output. Each input feature represents a 2D segment of the image generated in Step 2 with a defined size. In this process we employ striated 10-fold cross-validation to mitigate overfitting, and a high learning rate to speed up training. Adding additional omics data is straightforward, as the CNN head is agnostic to the depth of the data and will require a simple reorganization to the structure of the input space.
4. Validate CNN. To enable validation on data to which the trained model is completely blind, 10-20% of the initial samples are randomly selected and held out from the training set used to build the model. Once trained, performance is evaluated by using the model to predict age on the validation data and compared to the known age.

The algorithm used to assign genes to pixel positions within the image is one step that bears optimization. There are several published methods for constructing an image from RNAseq data and assigning genes to pixel position based on IGTD (used in our current clock; see Step 2 above). Examples include: (1) Deepsight, which assigns genes to pixel positions based on a t-distributed stochastic neighbor (t-SNE) projection; (2) Representation of Features as Images with Neighborhood Dependencies (REFINED), which minimized distance between similar pixels using Bayesian multidimensional scaling; or (3) OmicsMapNet, which clusters genes to pixel locations based on functional similarity. Each of these algorithms are, in principle, compatible with other forms of omics data. We evaluate the impact of using each method on prediction accuracy. In addition, we explore two novel variations on these methods: using MICe in place of (or in addition to) simple Spearman correlation, and combining functional information (e.g. gene ontology (GO) semantic similarity) with correlation in the pixel assignment algorithm. Finally, we empirically determined the minimum image size that provides accurate prediction.

The shift from a single-layer image (e.g. RNAseq alone) to multi-omics (e.g. RNAseq, proteomics, DNA methylation) provides the opportunity for additional optimization. We construct individual images for each layer of the predictor. In a sense, each omics layer is being represented as a different color channel, with the shift from single-layer to multi-layer conceptually mirroring the shift from a grayscale image to a color image. In the latter case, the relative position of pixels between layers becomes an important structural variable that will impact the ultimate prediction accuracy. In the case of positioning RNAseq and proteomics data, we place each protein in the same position as the corresponding gene and use correlation between gene and protein levels across samples to assign the gene-protein pair to a pixel position. The addition of a layer without a clear gene-protein relationship requires additional optimization, and we evaluate several methods for incorporating inter-layer correlation into the image-building process.

Aim 1.2. Optimize convolutional neural network. Our current image-based aging clock (FIG. 1A) was built as a proof-of-concept and is relatively unoptimized. First, the CNN receives a 74×130×1 image where each of the 9,620 pixels represents a unique gene present in the RNAseq dataset. We use an Adam optimization algorithm with a learning rate of 0.001 (without AMSGrad) and measure Mean Absolute Error (MAE) as our loss function. While we have examined some variants in each of these input parameters (image size, learning rate, loss function), we will explore a wider range of parameters as a simple optimization step. We can further refine the model by adding/removing CNN layers, feature mappings, or dense blocks. Second, we can also perform hyperparameter tuning, which can result in large performance gains. As a more fundamental performance optimization, we evaluate alternatives to CNNs. Increasingly Swin and other transformer models have shown the capacity to compete with CNN models for image pattern recognition and object detection. We built our initial clock using a CNN due to their past dominance in the image pattern recognition space and their relative ease to set up; however, given that our application is distinct from many other image-related tasks in the content of the data used to build the image, we evaluate transformer models as an alternative to CNNs. Finally, our current model has only been allotted 24 hours of total training (500 epochs per k-fold), which we increase as we refine many of the above parameters.

Deep learning modules have historically been “black boxes” that worked well at their given task, but were unable to report out what features were driving prediction accuracy. Recently, a method called SHapley Additive explanations (SHAP) has been developed to make machine learning algorithms more explainable by calculating contribution of each feature to prediction. SHAP is generalizable to any machine learning model and allows analyses of interaction between features. Once our model is trained, we employ SHAP as an optimization tool to identify important features and feature sets, and iteratively refine and reduce the features required to accurately predict biological age. We also use this approach to gain insight into which molecular processes are driving age prediction.

Pitfalls and alternatives. We have cleared the major technical hurdles and have constructed a first-order image-based aging clock that can accurately predict the age of a sample using RNAseq data alone. Given the multi-step nature of this clock concept, there are several axes along which we can optimize performance to identify a clock design with improved accuracy. Shifting from a single layer to a multi-layer omics clock presents additional challenges. While the CNN framework can easily accommodate multi-layer images, the relative position of pixels between layers presents additional optimization problems. Integrating cross-layer feature correlation into our current algorithm for determining relative pixel position optimizes prediction. In certain embodiments, other options may be incorporated, including pooling all features into a single image as an alternative to layering.

Aim 2. Construct image-based multi-omics remaining lifespan clock for aging mice. Our interest is in predicting a change in remaining lifespan for mice treated with an intervention starting at 18-22 months of age. A comparison of existing clocks, each built use biomarker panels derived from a single molecular layer (e.g. DNA methylation vs. telomere length), found both shared and independent influence on epidemiology and explanation of genetic variants across clocks. That suggests that, while some aspects of aging can be captured by single-layer clocks, those clocks are also likely missing information captured exclusively in other layers. A multi-omics clock can, in principle, capture that information. To our knowledge, there is no art that captures multiple omics layers in a minimally invasive manner that allows measurement of remaining lifespan. In this aim we have conducted a mouse aging study designed for multi-omics prediction of remaining lifespan. Our explicit goal is to capture as many layers of the multi-omics space as we can with minimally invasive tissue collection that allows unperturbed evaluation of remaining lifespan, as well as other health metrics.

Aim 2.1. Generate a multi-omics dataset From non-invasive measurement in mice. to achieve this goal, we collect a single blood sample from 80 male and 80 female 24-month-old C57BL/6J mice (The Jackson Laboratory) then follow the mice until end of life. Based on the one published example of a remaining lifespan clock—also constructed using C57BL/6J mice-and on pilot testing with datasets of different sample sizes while constructing our proof-of-principle image-based transcriptomic clock (FIG. 2A), a sample size of 160 animals is sufficient to build a robust biological age predictor, while smaller sample sizes (80-120) did not generalize well beyond the training set. In addition to blood, we also collect a skin sample (via tail tip and ear punch), feces, and urine, which will be stored for future analysis. While lifespan is our primary endpoint, we also measure body weight, frailty index (FI), rotarod performance, grip strength, and gait at baseline and every 3 months. Baseline metrics are used as an additional “physiological” layer for building the multi-omics clock, and subsequent measurements will be used as measurements of health trajectory, which we will use to further evaluate clock predictions at the end of the study.

Mice are ordered in cohorts of 30 to 40 scheduled a minimum of 2 weeks apart (and depending on when mice at the appropriate age are available for shipment) to allow sufficient time for sample processing and subsequent phenotyping. Mice are allowed to acclimate to the local environment for a minimum of 2 weeks before testing is initiated. Skin is collected as part of the animal identification process. Health and behavioral phenotyping is conducted prior to blood collection. We collect blood samples in tubes containing heparin to prevent coagulation and centrifuged for 10 minutes at 1000×g and 4° C. to pellet cells within 2 hours of collection. We collect and aliquot plasma, flash freeze, and stored at −80° C. until use. We use 55 μL for untargeted proteomics, metabolomics, and lipidomics by Dalton Bioanalytics using their one-shot multi-omics Omni-MS platform; 40 L for cell free RNAseq (cfRNAseq; Dalton Bioanalytics) following an optimized low-input volume protocol. We resuspend the cell pellet in PBS and evenly divided into 3 aliquots, one for DNA extraction (immediate; to BGI for whole-genome bisulfite sequencing following QC), one for RNA extraction (immediate; submitted to BGI for RNAseq following QC), and one for future analyses (flash frozen and stored at −80° C.). In some embodiments, multi-omics data can be collected by other means, for example standard non-integrated proteomics, lipidomics, and metabolomics pipelines.

Aim 2.2. Construct image-based multi-omics clock. As our primary objective, we construct a multi-omics clock using each data type (physiology, epigenome, transcriptome, proteome, metabolome, lipidome) as a separate image layer, using the optimized image building and CNN design determined in Aim 1 and remaining lifespan as the primary end point. Additional optimizations were conducted, guided by the clock performance. Since this is the first image-based clock constructed with more than two layers, we pay particular attention to feature number (i.e. image size) and relative placement within each layer. In addition to the full multi-layer clock, we build individual image-based clocks for each layer, and a “single-layer” multi-omics clock that incorporates all feature into a single image. We similarly build non-image based deep learning clocks in each category and compare performance across all clocks. This process allows us to fully vet (1) the importance of features from each layer, (2) the relative increase in performance for adding additional layers, and (3) the relative increase in performance for our image-based clock concept in the context of remaining lifespan prediction. Where available, we also compare the performance of our set of clocks to existing published clocks for which the selected features are both published and in our data set.

Pitfalls and alternatives. The major technical challenge in this Aim is to collect up to 7 omics layers (cell DNA methylation, cell transcriptome, plasma transcriptome, plasma proteome, plasma metabolome, plasma lipidome) from a single blood draw. Members of our team have experience collecting and analyzing each of these layers independently. Prior to ordering our first set of mice, we extensively validated our multi-omics pipeline in young C57BL/6J mice, including blood collection, sample preparation, quality control, and shipment, and collection of each type of data. While the sample quantities needed for each layer are achievable with a single blood collection per animal, we have identified alternative strategies for each of the sample preparation steps (e.g., RNA extraction for RNAseq) to improve yield if needed. For example, we can prioritize quality data generation in the other layers rather than expend resources generating data of dubious quality. Alternatively, if a single blood collection proves inadequate to generate quality data across layers, we can conduct a second blood collection two weeks after the first.

Aim 3. Validate the capability of the clock to predict lifespan extension in mice. A key validation test for any aging clock, and particularly in the area of drug discovery and repositioning, is the capacity to predict positive outcomes in response to interventions capable of extending healthy lifespan early in the treatment paradigm. Here we will validate the capability of image-based multi-omics clock to accurately predict remaining lifespan in response to three diet-based drug treatments known to extend lifespan relative to untreated controls: microencapsulated rapamycin (eRapa; Rapamycin Holdings), combined eRapa (Rapamycin Holdings, Inc) and metformin (MP Biomedicals), and 3-hydroxyanthranilic acid (3HAA; Ambeed, Inc). 20 male and 20 female 20-month-old C57BL/6 mice (The Jackson Laboratory) will be randomly assigned by cage to the following diets (drugs added to Purine 5K67 base diet by Research Diets, Inc):
Control (126 ppm Euradagit S100; microencapsulation material for eRapa)
126 ppm eRapa
126 ppm eRapa+1,000 ppm metformin
1000 ppm 3HAA+126 ppm Euradagit S100

Datasets are not yet available to conduct a detailed power analysis directly on the phenotype being measured (predicted remaining lifespan). Instead, we determined samples sizes based on published and internal lifespan studies conducted in C57BL/6 mice designed to provide 80% power to detect 10% change (or 95% power to detect 15% change) in lifespan per sex (α32 0.05, log-rank test). Starting at 24 months of age (4 months on test diets), they will be subjected to the same set of tests and sample collection outlined for the mice in the training study (Aim 2.1). All mice will be followed to end of life.

Multi-omics data will be collected for all blood samples as described (Aim 2.1) and used to predict remaining lifespan with each image-based clock (Aim 2.2). Correlation between predicted and measured remaining lifespan will be compared to assess each clock's accuracy. We will future examine the capacity to use predicted lifespan for the best performing clocks to predict health trajectories (frailty, grip strength, rotarod, behavior) of each mouse.

Data availability and future dataset expansion. Beyond our development of a novel aging clock concept, this work will produce a unique multi-omics mouse dataset. We will make all data from this work available on public data repositories and encourage its use as a benchmarking tool for future aging biomarker studies. As noted in the study design in Aim 2.1 and Aim 3, we will store samples that can be collected with minimal invasiveness (skin, feces, urine, excess plasma, excess blood cells) for future applications. Beyond this work, we will seek additional resources to expand the dataset to include other categories of data in the same mice (e.g. microbiome, urine and feces metabolome, skin multi-omics), increasing the value of the information collected here.

Impact. The most immediate impact of this work will be the validation of one (or more) aging clocks capable of predicting remaining lifespan in aging C57BL/6 mice. The availability of aged C57BL/6 mice through colonies at the National Institute on Aging (NIA), The Jackson Laboratory, and Charles River means that this tool can be immediately put into use as a primary tool for preclinical screening of candidate anti-aging drugs. While a broader study including mice at a range of ages would be more informative in some ways, we settled on predicting remaining lifespan specifically in 24-month-old mice as our initial target because that will allow a practical tool for drug screening to be put into practice on a compressed timeline (~2-3 years, rather than ~4-5 years for remaining lifespan to be measured starting in young mice). Our study is sufficiently large to allow, for the first time, detailed power analyses to be conducted specifically for clock-based predictive drug screens. We intend to pursue this application as soon as data is available (see Next Steps).

Beyond drug screening, this work will impact research on aging clocks in multiple more targeted ways. First, we assess the novel application of image-based CNNs pre-constructed image-like structure built from omics data at predicting phenotypes related to aging. Second, we conduct the most extensive examination to date of which omics layers are the most predictive for aging phenotypes, and to what extend incorporating multiple layers improves prediction accuracy. Third, we generate a unique multi-omics dataset that will serve the broader research field as a platform for aging clock benchmarking going forward.

The clock of the present invention may be used as a tool to accelerate preclinical drug screening. The study outlined in Aim 2 and Aim 3 provides proof-of-concept for the predictive capabilities of our image-based omics clocks for identifying drugs with potential to extend lifespan in a mammalian system. In parallel, there are several avenues that can refine our initial clock design. Multi-omics testing at the level proposed here is prohibitively expensive for expansion large-scaled drug testing. Using the data from this work, we can identify the minimum set of features necessary to accurately predict remaining lifespan and develop cost-effective targeted approaches to measure these features. Finally, more successful demonstrations of the value of non-invasive multi-omics in the context of remaining lifespan in 24-month-old mice will justify more extensive and longer-term studies starting in young mice, as well as expanding our scope to include genetically diverse populations, particularly the UM-HET3 mice used in the NIA Interventions Testing Program (ITP) and the diversity outbred (DO) mice used extensively for aging studies at The Jackson Laboratory.

### REFERENCES CITED

1 Xia X, Wang Y, Yu Z, Chen J, Han J-D J. Assessing the rate of aging to monitor aging itself. Ageing Res Rev 2021; 69:101350. https://doi.org/10.1016/j.arr.2021.101350.
2 Zhavoronkov A, Mamoshina P. Deep Aging Clocks: The Emergence of AI-Based Biomarkers of Aging and Longevity. Trends Pharmacol Sci 2019;40:546-9. https://doi.org/10.1016/j.tips.2019.05.004.
3 Gialluisi A, Santoro A, Tirozzi A, Cerletti C, Donati M B, de Gaetano G, et al. Epidemiological and genetic overlap among biological aging clocks: New challenges in biogerontology. Ageing Res Rev 2021;72:101502. https://doi.org/10.1016/j.arr.2021.101502.
4 Liu Z, Mao H, Wu C-Y, Feichtenhofer C, Darrell T, Xie S. A ConvNet for the 2020s. ArXiv Preprint 2022. https://doi.org/10.48550/arXiv.2201.03545.
5 Krizhevsky A, Sutskever I, Hinton GE. ImageNet Classification with Deep Convolutional Neural Networks. Advances in Neural Information Processing Systems 2012;25:.
6 Bello I, Fedus W, Du X, Cubuk ED, Srinivas A, Lin T-Y, et al. Revisiting ResNets: Improved Training and Scaling Strategies. Advances in Neural Information Processing Systems 2021;34:22614-27.
7 Liu Z, Lin Y, Cao Y, Hu H, Wei Y, Zhang Z, et al. Swin Transformer: Hierarchical Vision Transformer using Shifted Windows. 2021 IEEE/CVF International Conference on Computer Vision (ICCV) 2021:9992-10002. https://doi.org/10.1109/ICCV48922.2021.00986.
8 Shokhirev M N, Johnson A A. Modeling the human aging transcriptome across tissues, health status, and sex. Aging Cell 2021;20:e13280. https://doi.org/10.1111/acel.13280.
9 Johnson A A, Shokhirev M N, Lehallier B. The protein inputs of an ultra-predictive aging clock represent viable anti-aging drug targets. Ageing Res Rev 2021; 70:101404. https://doi.org/10.1016/j.arr.2021.101404.
10 Vijayakumar K A, Cho G-W. Pan-tissue methylation aging clock: Recalibrated and a method to analyze and interpret the selected features. Mech Ageing Dev 2022;204:111676. https://doi.org/10.1016/j.mad.2022.111676.
11 Sayed N, Huang Y, Nguyen K, Krejciova-Rajaniemi Z, Grawe A P, Gao T, et al. An inflammatory aging clock (iAge) based on deep learning tracks multimorbidity, immunosenescence, frailty and cardiovascular aging. Nat Aging 2021; 1:598-615. https://doi.org/10.1038/s43587-021-00082-y.
12 Meyer D H, Schumacher B. BiT age: A transcriptome-based aging clock near the theoretical limit of accuracy. Aging Cell 2021;20: e13320. https://doi.org/10.1111/acel.13320.
13 Galkin F, Mamoshina P, Kochetov K, Sidorenko D, Zhavoronkov A. DeepMAge: A Methylation Aging Clock Developed with Deep Learning. Aging Dis 2021;12:1252-62. https://doi.org/10.14336/AD.2020.1202.
14 Lu AT, Quach A, Wilson J G, Reiner AP, Aviv A, Raj K, et al. DNA methylation GrimAge strongly predicts lifespan and healthspan. Aging (Albany NY) 2019;11:303-27. https://doi.org/10.18632/aging.101684.
15 Schultz M B, Kane A E, Mitchell S J, MacArthur M R, Warner E, Vogel D S, et al. Age and life expectancy clocks based on machine learning analysis of mouse frailty. Nat Commun 2020;11:4618. https://doi.org/10.1038/s41467-020-18446-0.
16 Rutledge J, Oh H, Wyss-Coray T. Measuring biological age using omics data. Nat Rev Genet 2022. https://doi.org/10.1038/s41576-022-00511-7.
17 GTEx Consortium. The Genotype-Tissue Expression (GTEx) project. Nat Genet 2013;45:580-5. https://doi.org/10.1038/ng.2653.
18 The GTEx Consortium. The Genotype-Tissue Expression (GTEx) Project 2022. https://gtexportal.org/.
19 Takemon Y, Chick J M, Gerdes Gyuricza I, Skelly D A, Devuyst O, Gygi S P, et al. Proteomic and transcriptomic profiling reveal different aspects of aging in the kidney. Elife 2021; 10: e62585. https://doi.org/10.7554/eLife.62585.
20 Gerdes Gyuricza I, Chick J M, Keele G R, Deighan A G, Munger S C, Korstanje R, et al. Genome-wide transcript and protein analysis highlights the role of protein homeostasis in the aging mouse heart. Genome Res 2022;32:838-52. https://doi.org/10.1101/gr.275672.121.
21 Thompson M J, Chwiałkowska K, Rubbi L, Lusis A J, Davis R C, Srivastava A, et al. A multi-tissue full lifespan epigenetic clock for mice. Aging (Albany NY) 2018;10:2832-54. https://doi.org/10.18632/aging.101590.
22 Zhu Y, Brettin T, Xia F, Partin A, Shukla M, Yoo H, et al. Converting tabular data into images for deep learning with convolutional neural networks. Sci Rep 2021;11:11325. https://doi.org/10.1038/s41598-021-90923-y.
23 Sharma A, Vans E, Shigemizu D, Boroevich K A, Tsunoda T. DeepInsight: A methodology to transform a non-image data to an image for convolution neural network architecture. Sci Rep 2019; 9:11399. https://doi.org/10.1038/s41598-019-47765-6.
24 Bazgir O, Zhang R, Dhruba S R, Rahman R, Ghosh S, Pal R. Representation of features as images with neighborhood dependencies for compatibility with convolutional neural networks. Nat Commun 2020;11:4391. https://doi.org/10.1038/s41467-020-18197-y.
25 Ma S, Zhang Z. OmicsMapNet: Transforming omics data to take advantage of Deep Convolutional Neural Network for discovery. ArXiv Preprint 2019; arXiv:1804.05283:
26 Lundberg S M, Lee S-I. A Unified Approach to Interpreting Model Predictions. Advances in Neural Information Processing Systems 2017;30:.
27 Lundberg S M, Erion G G, Lee S-I. Consistent Individualized Feature Attribution for Tree Ensembles. ArXiv Preprint 2019. https://doi.org/10.48550/arXiv.1802.03888.
28 Zhou Z. Cell-free RNA Sequencing from Microliters of Unprocessed Serum. UC San Diego; 2017;2017.
29 Bitto A, Ito T K, Pineda V V, LeTexier N J, Huang H Z, Sutlief E, et al. Transient rapamycin treatment can increase lifespan and healthspan in middle-aged mice. Elife 2016;5:e16351. https://doi.org/10.7554/eLife.16351.
30 Neff F, Flores-Dominguez D, Ryan D P, Horsch M, Schröder S, Adler T, et al. Rapamycin extends murine lifespan but has limited effects on aging. J Clin Invest 2013;123:3272-91. https://doi.org/10.1172/JCI67674.
31 Harrison D E, Strong R, Sharp Z D, Nelson J F, Astle C M, Flurkey K, et al. Rapamycin fed late in life extends lifespan in genetically heterogeneous mice. Nature 2009;460:392-5. https://doi.org/10.1038/nature08221.
32 Miller R A, Harrison D E, Astle C M, Baur J A, Boyd A R, de Cabo R, et al. Rapamycin, but not resveratrol or simvastatin, extends life span of genetically heterogeneous mice. J Gerontol A Biol Sci Med Sci 2011;66:191-201. https://doi.org/10.1093/gerona/glq178.
33 Miller R A, Harrison D E, Astle C M, Fernandez E, Flurkey K, Han M, et al. Rapamycin-mediated lifespan increase in mice is dose and sex dependent and metabolically distinct from dietary restriction. Aging Cell 2014;13:468-77. https://doi.org/10.1111/acel.12194.
34 Strong R, Miller R A, Antebi A, Astle C M, Bogue M, Denzel MS, et al. Longer lifespan in male mice treated with a weakly estrogenic agonist, an antioxidant, an a-glucosidase inhibitor or a Nrf2-inducer. Aging Cell 2016;15:872-84. https://doi.org/10.1111/acel.12496.
35 Dang H, Castro-Portuguez R, Espejo L, Backer G, Freitas S, Spence E, et al. 3-hydroxyanthranilic acid-a new metabolite for healthy lifespan extension. BioRxiv 2021:2021.06.01.446651. https://doi.org/10.1101/2021.06.01.446651.
36 Sutphin, G. L. Unpublished Data n.d.
37 Nadon N L, Strong R, Miller R A, Harrison D E. NIA Interventions Testing Program: Investigating Putative Aging Intervention Agents in a Genetically Heterogeneous Mouse Model. EBioMedicine 2017;21:3-4. https://doi.org/ 10.1016/j.ebiom.2016.11.038.
38 Miller R A, Harrison D E, Astle C M, Floyd R A, Flurkey K, Hensley K L, et al. An Aging Interventions Testing Program: study design and interim report. Aging Cell 2007; 6:565-75. https://doi.org/10.1111/j.1474-9726.2007.00311.x.
39 Churchill G A, Gatti D M, Munger S C, Svenson K L. The Diversity Outbred mouse population. Mamm Genome 2012; 23:713-8. https://doi.org/10.1007/s 00335-012-9414-2.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A system for predicting a biological status of a mammal comprising a server that:
measures concentrations of one or more biomarkers in a serum sample;
receives an image comprising genetic data, wherein each pixel represents a unique gene;
applies a convolutional neural network to the image and the biomarker concentrations to analyze the interactions between the one or more biomarkers;
determines a biological status of a mammal from the analysis of the convolutional neural network; and
outputs one or more treatments for the mammal for the determined biological status.

2. The system of claim 1, wherein the biological status is biological age or a medical condition.

3. The system of claim 1, wherein the analysis comprises predicting phenotypes related the biological status.

4. The system of claim 1, wherein the system outputs an image-like structure on the basis of the analysis by the convolutional neural network.

5. The system of claim 1, wherein the serum sample is blood.

6. The system of claim 1, wherein the server further receives measurements of body weight, frailty index (FI), rotarod performance, grip strength, and gait.

7. The system of claim 1, wherein the server further generates a unique multi-omics dataset based on the convolutional neural network.

8. The system of claim 1, wherein the server further constructs a multi-omics clock using predetermined data types as a separate image layer.

9. The system of claim 8, wherein the predetermined data types comprise physiology, epigenome, transcriptome, proteome, metabolome, and lipidome.

10. The system of claim 1, wherein the biological status is determined based on one or more of: cell DNA methylation, cell transcriptome, plasma transcriptome, plasma proteome, plasma metabolome, and plasma lipidome.

11. A method for predicting a biological status of a mammal comprising:
measuring concentrations of one or more biomarkers in a serum sample;
receiving an image comprising genetic data, wherein each pixel represents a unique gene applying a convolutional neural network to the image and the biomarker concentrations to analyze the interactions between the one or more biomarkers;
determining a biological status of a mammal from the analysis of the convolutional neural network; and
treating the mammal for the determined biological status.

12. The method of claim 11, wherein the biological status is biological age or a medical condition.

13. The method of claim 11, wherein the analysis comprises predicting phenotypes related the biological status.

14. The method of claim 11, wherein the system outputs an image-like structure on the basis of the analysis by the convolutional neural network.

15. The method of claim 11, wherein the serum sample is blood.

16. The method of claim 11, further comprising receiving measurements of body weight, frailty index (FI), rotarod performance, grip strength, and gait.

17. The method of claim 11, further comprising generating a unique multi-omics dataset based on the convolutional neural network.

18. The method of claim 11, further comprising constructing a multi-omics clock using predetermined data types as a separate image layer.

19. The method of claim 18, wherein the predetermined data types comprise physiology, epigenome, transcriptome, proteome, metabolome, and lipidome.

20. The method of claim 11, wherein the biological status is determined based on one or more of: cell DNA methylation, cell transcriptome, plasma transcriptome, plasma proteome, plasma metabolome, and plasma lipidome.
